(19) United States
(12) Patent Application Publication
(10) Pub. No.: US 2006/0204015 A1
(43) Pub. Date: Sep. 14, 2006

(54) Noise cancellation module

(76) Inventors: Michael C. Ip, Fremont, CA (US); Yancy Chen, Campbell, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/078,390

(22) Filed: Mar. 14, 2005

(51) Int. Cl. A61F 11/06; H04B 15/00; G10K 11/16

(52) U.S. Cl. 381/71.1; 381/94.1

(57) ABSTRACT

A computer system having a noise cancellation module and a method of canceling noise using a noise cancellation module in a computer system are described. The computer system includes a processor for controlling operation of the computer system, a noise cancellation module coupled to the processor, and a memory coupled to the processor and storing operating instructions and at least one sequence of noise cancellation instructions. The noise cancellation instructions cause the processor to generate cancellation audio signals responsive to receipt of audio signals from the noise cancellation module, and transmit the generated cancellation audio signals to the noise cancellation module. The method includes sensing, by the noise cancellation module, an audio signal generated by the noise-generating source and causing the processor to generate a noise cancellation signal responsive thereto. The noise cancellation module generates an audio signal based on the generated noise cancellation signal from the processor.

### FIELD OF THE INVENTION

The present invention relates to a noise cancellation module.

### BACKGROUND

Current computer systems have a number of devices many of which generate an audible level of noise to a user of the computer system. For example, many systems have one or more fans for cooling and motors for rotating media. Each of these devices generates noise which is heard by the human ear.

In order to overcome the generated computer system noise, many users increase the output volume of the computer system or of music, e.g., music being generated by the computer system. Because of human ear functionality, the louder volume makes the generated computer system noise less distinct. Disadvantageously, the generated noise remains in the background and may be heard during soft or lower volumes portions of the music or other output audio.

### SUMMARY

The present invention provides a noise cancellation module.

An apparatus aspect includes a computer system having a noise cancellation module. The computer system includes a processor for controlling operation of the computer system, a noise cancellation module coupled to the processor, and a memory coupled to the processor and storing operating instructions and at least one sequence of noise cancellation instructions. The noise cancellation instructions, when executed by the processor, cause the processor to receive audio signals from the noise cancellation module, generate cancellation audio signals, and transmit the generated cancellation audio signals to the noise cancellation module.

A method aspect of canceling noise generated by a noise-generating source of a computer system having a noise cancellation module operatively coupled with a processor includes sensing, by the noise cancellation module, an audio signal generated by the noise-generating source. A sequence of instructions executed by the processor cause the processor to generate a noise cancellation signal in response to receiving the sensed audio signal from the noise cancellation module. An audio signal is generated, by the noise cancellation module, based on the generated noise cancellation signal from the processor.

Still other advantages of the embodiments will become readily apparent to those skilled in the art from the following detailed description, wherein the preferred embodiments are shown and described, simply by way of illustration of the best mode contemplated of carrying out the invention. As will be realized, the invention is capable of other and different embodiments, and its several details are capable of modifications in various obvious respects, all without departing from the invention.

### DESCRIPTION OF THE DRAWINGS

The present invention is illustrated by way of example, and not by limitation, in the figures of the accompanying drawings, wherein elements having the same reference numeral designations represent like elements throughout and wherein:

FIG. 1 is a high level functional block diagram of a computer system useable in conjunction with an embodiment;

FIG. 2 is a high level block diagram of an embodiment installed in a computer system; and

FIG. 3 is a functional flow diagram of an embodiment.

### DETAILED DESCRIPTION

In contrast with the above-described approaches, the mechanism of the present invention includes a noise cancellation module and instructions for generating cancellation audio signals responsive to sensed audio signals.

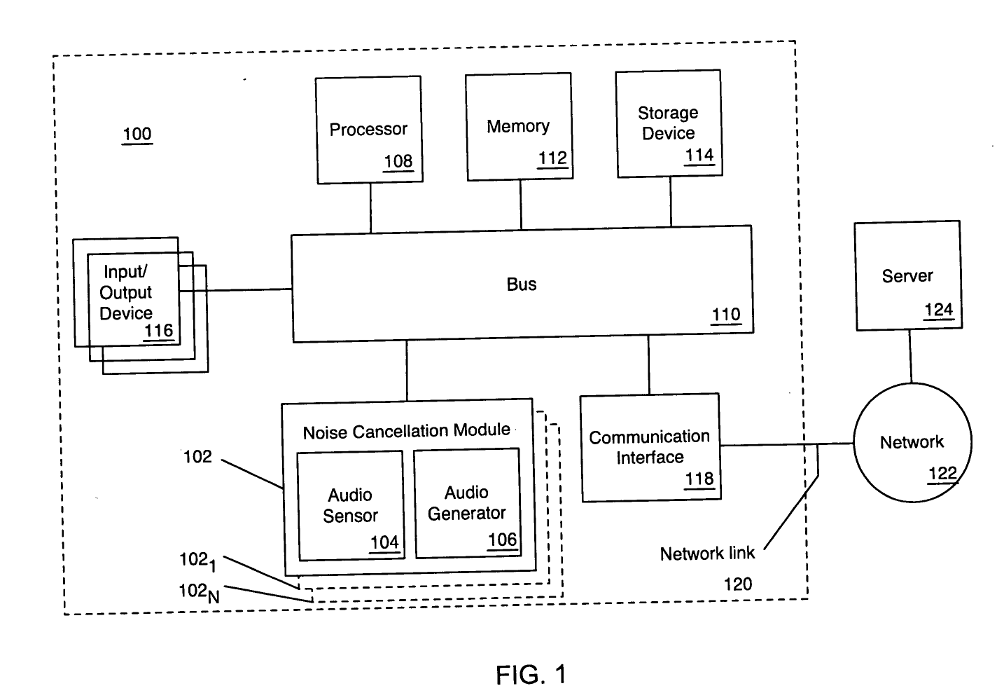

In accordance with an embodiment of the present invention, FIG. 1 depicts a computer system 100 having a noise cancellation module 102 installed therein. As depicted in FIG. 1 using dashed lines, there may be more than one noise cancellation module 102₁-102_{N}; however, for the sake of simplicity only a single noise cancellation module 102 will be described with respect to FIG. 1. Noise cancellation module 102 includes an audio sensor 104 for sensing audio signals, e.g., a microphone, and an audio generator 106 for generating audio signals, e.g., a speaker. In one embodiment, audio generator 106 is a low cost buzzer speaker. In operation, noise cancellation module 102 receives sensed audio signals via audio sensor 104 and provides the signals to a processor 108 for processing, i.e., generation of cancellation audio signals. Noise cancellation module 102 receives the generated cancellation audios signals from processor 108 and drives audio generator 106 to generate the audio signals. That is, audio sensor 104 senses the audio signals while audio generator 106 produces cancellation audio signals to cancel the sensed audio signals.

Computer system 100 includes a bus 110 or other communication mechanism for communicating information, and processor 108 coupled with the bus 110 for processing information. Processor 108 is the computer system processor in control of operation of the computer system, i.e., the processor is the central processing unit of computer system 100 executing, among other things, operating system instructions. For example, in the FIG. 1 embodiment, processor 108 receives sensed audio signals from noise cancellation module 102 (specifically audio sensor 104), processes the audio signals to generate cancellation audio signals, and transmits the generated cancellation audio signals to the noise cancellation module (specifically, audio generator 106).

Computer system 100 also includes a memory 112, such as a random access memory (RAM) or other dynamic storage device, coupled to bus 110 for storing audio signal data, cancellation audio signal data, and instructions to be executed by processor 108. Memory 112 also may be used for storing temporary variables or other intermediate information during execution of instructions to be executed by processor 108. Computer system 100 further includes a storage device 114, such as a magnetic disk or optical disk, coupled to bus 110 for storing audio signal data, cancellation audio signal data, and instructions.

Computer system 100 may be coupled via bus 110 to one or more input/output devices 116, such as a display, a keyboard, a mouse, etc. Embodiments are related to the use of computer system 100, such as the depicted system of FIG. 1, to sense audio signals and generate cancellation audio signals responsive to the sense audio signals. According to one embodiment, cancellation audio signals are generated by computer system 100 in response to processor 108 executing sequences of instructions contained in memory 112 in response to input audio signals received via noise cancellation module 102. Such instructions may be read into memory 112 from another computer-readable medium, such as storage device 114.

However, the computer-readable medium is not limited to devices such as storage device 114. For example, the computer-readable medium may include a floppy disk, a flexible disk, a hard disk, a magnetic tape, or any other magnetic medium, a CD-ROM, any other optical medium, punch cards, paper tape, any other physical medium with patterns of holes, a RAM, a PROM, an EPROM, a FLASH-EPROM, any other memory chip or cartridge, a carrier wave embodied in an electrical, electromagnetic, infrared, or optical signal, or any other medium from which a computer can read. Execution of the sequences of instructions contained in memory 112 causes processor 108 to perform the process steps described below. In alternative embodiments, hard-wired circuitry may be used in place of or in combination with computer software instructions to implement the invention. Thus, embodiments of the invention are not limited to any specific combination of hardware circuitry and software.

Computer system 100 also includes a communication interface 118 coupled to bus 110. Communication interface 118 provides two-way data communication as is known. For example, communication interface 118 may be an integrated services digital network (ISDN) card, a digital subscriber line (DSL) card, or a modem to provide a data communication connection to a corresponding type of telephone line. As another example, communication interface 118 may be a local area network (LAN) Card to provide a data communication connection to a compatible LAN. Wireless links may also be implemented. In any such implementation, communication interface 118 senses and receives electrical, electromagnetic, or optical signals which carry digital data streams representing various types of information. Of particular note, the communications through interface 118 may permit transmission or receipt of audio signals and cancellation audio signals. For example, two or more computer systems 100 may be networked together in a conventional manner with each using a communication interface 118.

A network link 120 typically provides data communication through one or more networks to other data devices. For example, a network link 120 may provide a connection through local network 122 to a server 124. Network 122 includes data communications services provided through the worldwide packet data communication network now commonly referred to as the Internet. The signals through network 122 and on network link 120 and through communication interface 118, which carried the digital data to and from computer system 100, are exemplary forms of carrier waves transporting information.

Computer system 100 can send messages and receive data, including program code, through the network 122, network link 120, and communication interface 118. In the Internet example, server 124 might transmit a requested code for an application program through network 122 and communication interface 118. In accordance with the present embodiments, one such downloaded application provides for generation of cancellation audio signals responsive to receipt of audio signals by processor 108.

The received code may be executed by processor 108 as it is received, and/or stored in storage device 114, or other storage for later execution. In this manner, computer system 100 may obtain application code in the form of a carrier wave.

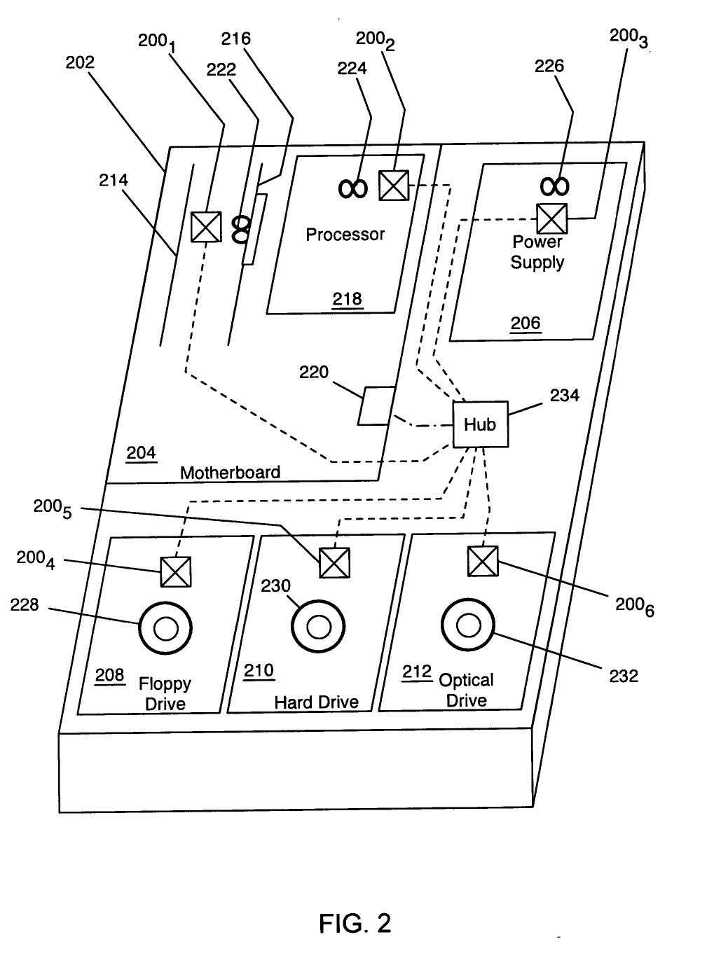

FIG. 2 depicts a high level block diagram of multiple noise cancellation modules 2001₋₆ installed in a computer system 202. Computer system 202 includes functionality similar to computer system 100 (FIG. 1). Computer system 202 includes a motherboard 204 including functionality of bus 110 (FIG. 1), a power supply 206 for providing power to components of computer system 202, and a floppy drive 208, a hard drive 210, and an optical drive 212 for providing functionality similar to storage device 114 (FIG. 1). Motherboard 204 includes a sound card 214 for generating and/or sensing audio signals, a video card 216 for generating and/or receiving video signals, and a processor 218 providing functionality similar to processor 108 (FIG. 1). Further, motherboard 204 includes an input/output port 220 for sending and receiving signals to/from processor 218.

Although not depicted in FIG. 2, power supply 206, floppy drive 208, hard drive 210, and optical drive 212 are all connected to motherboard 204. In different embodiments, each of the components may be interconnected to each other or to selected subsets in different manners.

Many of the FIG. 2 components produce noise detectable by the user of computer system 202. Several of the components depicted in FIG. 2 have fans for providing cooling and/or heat dissipation which generate noise, e.g., video card 216 has a fan 222, processor 218 has a fan 224, and power supply 206 has a fan 226. Additionally, several of the FIG. 2 components have motors which generate noise, e.g., floppy drive 208 has a motor 228, hard drive 210 has a motor 230, and optical drive 212 has a motor 232.

In alternative embodiments, different components of computer system 202 (not shown) generate noise detectable by a user of the system.

In accordance with one embodiment, a noise cancellation module 2001₋₆ is positioned adjacent to a noise-generating source, e.g., fans 222, 224, and 226 and motors 228, 230, and 232. Each noise cancellation module 2001-6 is connected with input/output port 220 via a hub 234. In an alternate embodiment, each noise cancellation module 2001-6 is directly connected to input/output port 220 without the need for hub 234. Hub 234 may be needed if the number of noise cancellation modules 200₁₋₆ exceeds the input/output capabilities of input/output port 220.

In a further alternate embodiment, an additional hub 234 may be added to computer system 202 if the capabilities of a first hub 234 are exceeded. Hub 234 may be included as a separate component of computer system 202 or integrated as a part of motherboard 204, e.g., integrated as part of input/output port 220 or as a separate component of the motherboard. Hub 234 may be a serial, parallel, USB, or other similar device.

It will be understood that more or less noise cancellation modules may be used in conjunction with a computer system depending on the number of noise-generating sources.

Each audio sensor 104 of the respective noise cancellation modules 2001₋₆ receives audio signals representing noise generated by the corresponding noise-generating source adjacent to which the sensor is positioned. Noise cancellation modules 2001₋₆ transmit the sensed audio signals to processor 218 via input/output port 220 and hub 234. As described above, processor 218, upon receipt of sensed audio signals, calculates cancellation audio signals for generation by audio generator 106 in order to cancel the sensed audio signals. Processor 218 transmits the generated cancellation audio signals to audio generator 106 via input/output port 220 and hub 234. Responsive to receipt of the generated cancellation audio signals, audio generator 106 generates a sound wave based on the received generated cancellation audio signals.

In this manner in an embodiment, the existing processor of the computer system is used to generate noise cancellation signals. Additional processors and/or digital signal processors (DSP) are not required for operation of the embodiment. Processor 218 of computer system 202 is the same processor which executes operating system and application software instructions and controls operation of the computer system.

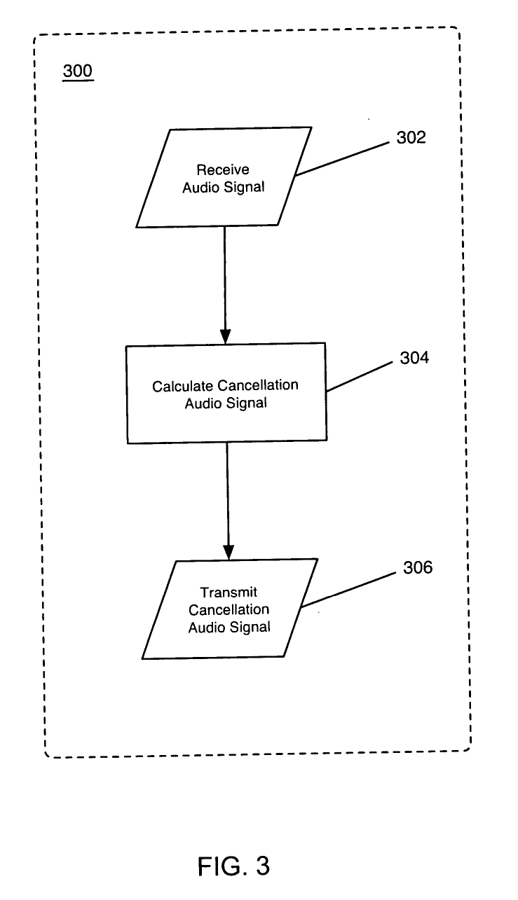

FIG. 3 depicts a functional process flow view of a sequence of instructions executed by processor 218 according to an embodiment. As depicted in FIG. 3, process flow 300 represents a portion of the sequence of instructions executed by processor 218 in the course of normal operation. Processor 218 executes process flow 300 as a background task of computer system 202 in conjunction with operating system and application instructions. The flow of control begins at step 302 in response to processor 218 receiving audio signals from noise cancellation module 200 via input/output port 220. During step 302, the received audio signal may be stored in a portion of processor 218 or in a memory (not shown in FIG. 2, but similar to memory 112 of FIG. 1).

After the audio signal is received by processor 218, the flow proceeds to step 304 wherein processor 218 calculates a noise cancellation signal based on the received audio signal from step 302. During step 304, processor 218 calculates a noise cancellation signal capable of canceling the received audio signal when generated by audio generator 106 in noise cancellation module 200. In an embodiment, processor 218 calculates the noise cancellation signal by inverting the phase of the received sensed audio signal.

In an embodiment, processor 218 samples the incoming audio signal, e.g., a digitized noise signal detected by noise cancellation module 200, and generates an equal amplitude, opposite phase wave form. In a further specific embodiment, the generated wave form is 100% inverted from the incoming audio signal. After the generated wave form is amplified and output via audio generator 106, e.g., a speaker, the wave form cancels out the existing noise. In a further embodiment, the generated wave form reduces the existing noise due to a slight time shift during the generation of the wave form and transmission time between the noise cancellation module 200 and processor 218.

After the noise cancellation signal is determined by processor 218, the flow proceeds to step 306 and the processor transmits the noise cancellation signal to noise cancellation module 200 via input/output port 220 and hub 234. Audio generator 106 of noise cancellation module 200 produces an audio wave responsive to receipt of, and based on, the noise cancellation signals.

In one embodiment, audio generator 106 is positioned farther from a noise-generating source, e.g., fans 222, 224, and 226 and motors 228, 230, and 232, than audio sensor 104.

It will be readily seen by one of ordinary skill in the art that the embodiments fulfills one or more of the advantages set forth above. After reading the foregoing specification, one of ordinary skill will be able to affect various changes, substitutions of equivalents and various other aspects of the embodiments as broadly disclosed herein. It is therefore intended that the protection granted hereon be limited only by the definition contained in the appended claims and equivalents thereof.

For example, although each of the components with respect to FIG. 2 above is described and depicted as being within computer system 202, it will be understood that external noise generating sources may be compensated for in a similar manner. Further, although audio sensor 104 and audio generator 106 are described as being part of a noise cancellation module 102, it will be understood that the audio sensor and audio generator may be positioned as separate components, as well as, separately connected to processor 108.

What is claimed is:

1. A computer system having a noise cancellation module, comprising:
a processor for controlling operation of the computer system;
a noise cancellation module coupled to the processor; and
a memory coupled to the processor and storing operating instructions and at least one sequence of noise cancellation instructions which, when executed by the processor, cause the processor to generate cancellation audio signals responsive to receipt of audio signals from the noise cancellation module, and transmit the generated cancellation audio signals to the noise cancellation module.

2. The system as claimed in claim 1, the noise cancellation module further comprising:
an audio sensor for receiving audio signals; and
an audio generator for generating audio signals.

3. The system as claimed in claim 2, wherein the audio generator is positioned farther away from a noise-generating source than the audio sensor.

4. The system as claimed in claim 1, wherein the operating instructions include at least one sequence of operating system instructions for controlling operation of the computer system.

5. The system as claimed in claim 1, wherein the noise cancellation module is coupled to the processor via at least one of an input/output port.

6. The system as claimed in claim 1, wherein the noise cancellation module is coupled to the processor via a motherboard-based input/output port.

7. The system as claimed in claim 6, wherein the motherboard-based input/output port includes at least one of a serial port, a parallel port a universal serial bus-type port, and a FireWire-type port.

8. The system as claimed in claim 1, further comprising:
a hub connecting the noise cancellation module to the processor.

9. A computer system having one or more noise cancellation modules, comprising:
a processor for controlling operation of the computer system;
one or more noise-generating sources operatively coupled with the computer system;
one or more noise cancellation modules each positioned adjacent one of the one or more noise-generating sources and operatively coupled with a processor of the computer system, the noise cancellation modules each communicating sensed audio signals to the processor and cancellation audio signals from the processor; and
a memory coupled to the processor and storing operating instructions.

10. The computer system as claimed in claim 9, the noise cancellation module further comprising at least one of an audio sensor and an audio generator.

11. The computer system as claimed in claim 9, wherein the memory further includes at least one sequence of noise cancellation instructions which, when executed by the processor, cause the processor to generate cancellation audio signals responsive to receipt of audio signals from the noise cancellation module, and transmit the generated cancellation audio signals to the noise cancellation module.

12. The computer system as claimed in claim 9, further comprising:
a hub connecting the one or more noise cancellation modules to the processor.

13. The computer system as claimed in claim 9, wherein the one or more noise cancellation modules are coupled to the processor via a motherboard-based input/output port.

14. The computer system as claimed in claim 13, wherein the motherboard-based input/output port includes at least one of a serial port, a parallel port a universal serial bus-type port, and a FireWire-type port.

15. A method of canceling noise generated by a noise-generating source of a computer system having a noise cancellation module operatively coupled with a processor, comprising the steps of:
sensing, by the noise cancellation module, an audio signal generated by the noise-generating source;
executing a sequence of instructions by the processor causing the processor to generate a noise cancellation signal in response to receiving the sensed audio signal from the noise cancellation module; and
generating an audio signal, by the noise cancellation module, based on the generated noise cancellation signal from the processor.

16. The method as claimed in claim 15, wherein the noise cancellation module further includes an audio sensor and an audio generator.

17. The method as claimed in claim 15, further comprising the step of:
executing, by the processor, at least one sequence of instructions for controlling operation of the computer system.

18. The method as claimed in claim 15 wherein the executing step includes instructions causing the processor to generate an inverted phase audio signal from the sensed audio signal.

19. The method as claimed in claim 15, wherein the processor receives the sensed audio signals via an input/output port of the computer system.

20. The method as claimed in claim 15, wherein a hub operatively couples the noise cancellation module to the processor.
